(19) United States
(12) Patent Application Publication
(10) Pub. No.: US 2025/0037801 A1
(43) Pub. Date: Jan. 30, 2025

(54) METHOD FOR PREDICTING GENE EDITING ACTIVITY BY DEEP LEARNING AND USE THEREOF

(71) Applicant: Center for Excellence in Brain Science and Intelligence Technology, Chinese Academy of Sciences, Shanghai (CN)

(72) Inventors: Yidi SUN, Shanghai (CN); Changyang ZHOU, Shanghai (CN); Leilei WU, Shanghai (CN)

(73) Assignee: Center for Excellence in Brain Science and Intelligence Technology, Chinese Academy of Sciences, Shanghai (CN)

(21) Appl. No.: 18/843,609

(22) PCT Filed: Mar. 4, 2022
(86) PCT No.: PCT/CN2022/079264

§ 371 Date: Sep. 3, 2024

(51) Int. Cl. G16B 40/20; G16B 30/00

(52) U.S. Cl. CPC G16B 40/20; G16B 30/00

(57) ABSTRACT

The present invention provides a model or tool for predicting the editing efficiency of sgRNA in a CRISPR/Cas gene editing system, in particular a CRISPR/dCas epigenetic editing system, a training and prediction method thereof, and a related computer system, computer storage medium, and application. In particular, one or more sgRNA and target gene related epigenetic features are added to an input of the model.

### FIELD

The present invention relates to gene editing and deep learning, and specifically to a method for predicting the epigenetic editing activity of a CRISPR/Cas system based on deep learning.

### BACKGROUND

The CRISPR/Cas system is a powerful DNA-editing tool that has become one of the research highlights in recent years, and it can flexibly target specific genomic sequences and result in DNA double-strand breaks (DSBs). Guided by a specific small guide RNA (sgRNA), the Cas nuclease in the CRISPR/Cas system binds and functions adjacent to a specific genomic site to cleave DNA double strands. The broken DNA is repaired through the DNA damage repair pathway of the cell, including non-homologous end joining and homology-mediated repair modes, which generate gene knock-in or knock-out, either specifically or randomly, depending on whether there is a DNA template.

Mutation in the Cas protein can result in a Cas protein that loses its cleavage activity (dCas). A dCas protein complex fused with a transcription repressor or transcription activator will be targeted to a specific region of the genome under the guidance of a specific sgRNA, resulting in inhibition (CRISPRi) or activation (CRISPRa) of transcription of the target gene, thus allowing for editing of transcripts without altering the DNA sequence [Luke A Gilbert et al., CRISPR-mediated modular RNA-guided regulation of transcription in eukaryotes, Cell. 2013 Jul. 18: 154 (2): 442-51]. Nuñez, James K et al. have further proposed a CRISPRoff/on editing system, which can increase or decrease DNA methylation and inhibitory histone modifications by fusing the dCas protein with a methylase or demethylase, thereby achieving the purpose of reducing specific gene expression [James K Nuñez et al., Genome-wide programmable transcriptional memory by CRISPR-based epigenome editing, Cell. 2021 Apr. 29: 184 (9): 2503-2519.e17]. The transient expression of CRISPRoff can initiate DNA methylation at specific genomic sites to produce gene silencing, a state that can be stably maintained during cell division and differentiation of stem cells into neurons. Moreover, demethylation occurs and the expression state of silenced genes is restored under the action of the CRISPRon editing system. The proposal of epigenetic editing systems such as CRISRPi/a/off/on provides a convenient approach for exploring epigenetic research.

In CRISPR-related editing systems, the editing efficiency of certain sgRNAs targeting the same gene is superior to that of other sequences, and this is because the specificity of the CRISPR editing system depends on the sgRNA sequence [John G Doench et al., Rational design of highly active sgRNAs for CRISPR-Cas9-mediated gene inactivation, Nat Biotechnol. 2014 Dec.: 32 (12): 1262-7]. In order to select highly efficient sgRNAs for the CRISPR editing system, researchers have designed a series of sgRNA libraries for screening and developed prediction algorithms [Hui Kwon Kim et al., SpCas9 activity prediction by DeepSpCas9, a deep learning-based model with high generalization performance Sci Adv. 2019 Nov. 6: 5 (11): eaax9249]. These prediction tools modelled a large amount of experimental data based on machine learning models in an attempt to select out sgRNAs with high on-target activity and low off-target effect through the model. However, although many models have achieved good performance on their training datasets, predictions on other datasets are not accurate [E A Moreb et al., Genome dependent Cas9/gRNA search time underlies sequence dependent gRNA activity, Nat Commun. 2021 Aug. 19: 12 (1): 5034]. One of the reasons may be that these algorithms rely only on sequence features of sgRNAs and target sequences. Moreover, existing prediction models are usually designed based on CRISPR/Cas gene editing systems, and few prediction tools have been developed separately for epigenetic editing tools such as CRISPR/dCas.

The present invention addresses the above needs by providing an epigenetic editing prediction tool for predicting CRISPR/dCas epigenetic editing systems based on a deep learning model.

### SUMMARY

The present disclosure provides a tool for predicting the editing efficiency of sgRNA in a CRISPR/Cas gene editing system, especially a CRISPR/dCas epigenetic editing system, and a production method and application thereof. The inventors found that adding specific epigenetic features to the input of the algorithm can effectively improve the accuracy of sgRNA editing efficiency prediction in the model, obtain better generalization performance and shorten the time required for training. In some embodiments, one or more of the following epigenetic features are added to the input of the algorithm: distance between the transcription start site (TSS) and the sgRNA target site, DNA methylation level, RNA expression level and chromosome accessibility. Preferably, all four epigenetic features are added to the input of the algorithm.

In one aspect, the present disclosure provides a model training method for predicting the gene editing activity of sgRNA in a CRISPR/Cas system, comprising the following steps:
1) constructing or acquiring a dataset comprising sgRNAs and editing activity data thereof for model training, for example, for prediction of a CRISPR/dCas system such as a CRISPRoff epigenetic system, the dataset may include a CRISPRoff_tiling dataset, wherein the CRISPRoff_tiling dataset is derived from a CRISPRoff screening experiment in HEK293T cells consisting of 520 genes and containing 111,638 targeting sgRNAs;
2) encoding the sgRNA sequences and one or more epigenetic features in the sample dataset utilizing specific coding methods, rendering them available as inputs to a neural network, wherein the DNA sequences (preferably 40 bases in length) of the genomic regions associated with the sgRNA sequences in the training sample are converted into a binary matrix according to the types of each base in the DNA sequences, using one-hot encoding method: moreover, one or more of the following epigenetic features are converted into a continuous variable matrix: distance between transcription start site (TSS) and sgRNA target site, DNA methylation level, RNA expression level and chromosome accessibility, and: the binary matrix and the continuous variable matrix are concatenated as an input matrix for training;
3) building a convolutional neural network model which includes five parallel convolution layers and three cascaded fully connected layers behind the convolution layers, wherein the five convolution layers extract features from the input matrix in parallel, and the outputs from each convolution layer are concatenated as inputs to the fully connected layers: preferably, the convolutional neural network model further includes at least one of the following: a pooling layer between the convolution layers and the fully connected layers, an input layer using linear activation functions, a drop-out function behind the convolution layers, and a drop-out function behind the fully connected layers;
4) dividing the dataset prepared in step 1) into a training set and a testing set, followed by training the convolutional neural network model of step 3) using the input matrix and output ground truth of each sample in the training set, and determining the output accuracy of the trained model using the testing set, finally terminating the training when the output accuracy meets the requirements, thereby obtaining the trained model parameters.

In another aspect, the disclosure provides a method for predicting the gene editing activity of sgRNA in a CRISPR/Cas system based on deep learning, comprising the following steps:
1) establishing a prediction model for predicting the gene editing activity of CRISPR/dCas sgRNA based on a dataset comprising sgRNAs and editing activity data thereof, one or more epigenetic features, and a convolutional neural network model;
2) transforming the sgRNA sequence to be tested and related epigenetic features thereof into an input matrix suitable for the prediction model, and inputting it into the prediction model to obtain a predicted value of sgRNA activity.

In some embodiments, the above-mentioned model for predicting the gene editing activity of CRISPR/dCas sgRNA includes a classification model or regression model based on convolutional neural network (CNN).

In some embodiments, in response to a CNN-based classification model as the training target, an output ground truth of the training sample is labelled, for example, those with significant editing effects can be labelled as “1”, and the remaining ones can be labelled as “0”.

In some embodiments, in response to a CNN-based regression model as the training target, the output ground truth of the training sample, i.e., the gene editing efficiency, is expressed as a phenotype score (γ). The higher absolute value of the phenotype score represents a more efficient editing, which is calculated as follows:

$$phenotype score ( γ ) = \frac{{Log}_{2} sgRNA enrichment}{fold difference} ,$$

wherein the “sgRNA enrichment” represents the fold change after the CRISPR/dCas experiment compared to the beginning of the experiment, and the “fold difference” represents cell proliferation folds during the CRISPR/dCas experiment, after dividing the two, one can get the degree to which the sgRNA at each site is knocked-down (for example, in CRISPRi/CRISPRoff editing systems) or activated (for example, in CRISPRa editing systems), which characterizes the editing efficiency of sgRNA.

In some embodiments, the epigenetic features of DNA methylation level, RNA expression level and chromosome accessibility are quantified by whole-genome bisulfite sequencing data, RNA-seq data, and ATAC-seq data, respectively.

In some embodiments, genomic target sequences of 40 nucleotides in length are used as input to the model, which contains an upstream sequence of 9 base-pairs (bp), a protospacer sequence of 20 base-pairs (bp), a PAM sequence of 3 base-pairs (bp) and a downstream sequence of 8 base-pairs (bp). The protospacer sequence of 20 base-pairs (bp) corresponds to the sgRNA sequence in the training sample.

In some embodiments, the dataset is divided into a training set and a testing set at a ratio of 9:1. Optionally, in order to reduce the impact from data deviation, step 4) is repeated multiple times, such as 20 times, and the training set and testing set are randomly re-divided each time to ensure the stability of the model.

In some embodiments, each of the five convolution layers uses 30 filters, and the kernel size of each convolution layer is 1, 2, 3, 4, and 5, respectively.

In some embodiments, the three fully connected layers contain 80, 60 and 40 units, respectively.

In some embodiments, the drop-out function has a drop-out rate of 0.4.

In yet another aspect, provided herein is a computer/computational system for assisting users in predicting the gene editing activity, comprising:
one or more processors; and
one or more memories configured to store a series of computer-executable instructions,
wherein, when the series of computer-executable instructions are executed by the one or more processors, the one or more processors are allowed to perform the method as described above.

In a further aspect, provided herein is a non-transitory computer-readable storage medium, wherein a series of computer-executable instructions are stored on the non-transitory computer-readable storage medium, and when the series of computer-executable instructions are executed by one or more computing devices, the one or more computing devices are allowed to perform the method as described above.

Specifically, the present disclosure relates to the following embodiments:
1. A model training method for predicting the editing activity of sgRNA in a CRISPR/dCas gene editing system, comprising:
constructing or acquiring a dataset comprising sgRNAs and editing activity data thereof for model training;
constructing inputs for training based on sgRNA sequences and one or more epigenetic features in the sample dataset;
building a convolutional neural network (CNN) model;
dividing the dataset into a training set and a testing set, training the CNN model using the input matrix and output ground truth of each sample in the training set, and determining the output accuracy of the trained model using the testing set; and
terminating the training when the output accuracy is satisfied, thereby obtaining model parameters that have been trained.
2. The method according to embodiment 1, wherein the dataset comprises at least one of the following: a CRISPRoff_tiling dataset, a CRISPRoff_genomeA dataset, a CRISPRi_intergrate dataset, a CRISPRi_genome dataset, a CRISPRi_CRISPRoffsource dataset, a hCRISPRiV2 dataset, a hCRISPRav2 dataset, or a CRISPRa_intergrate dataset. Preferably, for CRISPR/dCas editing systems that inhibit gene expression, the dataset is a CRISPRoff_tiling dataset, and for CRISPR/dCas editing systems that activate gene expression, the dataset is a hCRISPRiV2 dataset.
3. The method according to embodiment 2, wherein the CRISPRoff_tiling dataset is based on the CRISPRoff screening experiments in HEK293T cells, comprising 520 genes and 111,638 targeting sgRNAs.
4. The method according to embodiment 2, wherein the hCRISPRiV2 dataset is based on the CRISPRa screening experiments in K562 cells, comprising 198,757 targeting sgRNAs.
5. The method according to embodiment 2, wherein datasets other than the selected dataset are used to test the generalization of the model.
6. The method according to any one of the preceding embodiments, wherein, constructing the DNA sequence of the genomic region associated with the sgRNA sequence in the training sample as a binary matrix according to the base type at each nucleotide position using the one-hot encoding method;
constructing the one or more epigenetic features at each nucleotide position as a continuous variable matrix; and
concatenating the binary matrix and the continuous variable matrix as an input matrix for training.
7. The method according to embodiment 6, wherein the DNA sequence has a length of 30-40 base-pairs.
8. The method according to embodiment 7, wherein the DNA sequence has a length of 40 base-pairs and comprises an upstream sequence of 9 base-pairs, a protospacer sequence of 20 base-pairs, a PAM sequence of 3 base-pairs, and a downstream sequence of 8 base-pairs.
9. The method according to embodiment 8, wherein the protospacer sequence of 20 base-pairs corresponds to or is complementary to the sgRNA sequence in the training sample.
10. The method according to any one of the preceding embodiments, wherein the CNN model includes five parallel convolution layers and three cascaded fully connected layers after the convolution layers.
11. The method according to embodiment 10, wherein the five convolution layers extract features from the input matrix in parallel, and the outputs from each convolution layer are concatenated as inputs to the fully connected layers.
12. The method according to any one of the preceding embodiments, wherein the CNN model further includes at least one of the following:
a pooling layer between the convolution layer and the fully connected layer, an input layer using linear activation functions, a drop-out function behind the convolution layers, or a drop-out function behind the fully connected layers.
13. The method according to any one of the preceding embodiments, wherein the CNN model includes a classification model or regression model.
14. The method according to embodiment 13, further comprising:
in response to a classification model as the CNN model, labelling an output ground truth of each training sample, of which, those with editing effects greater than the threshold are labelled as “1”, and the remaining ones are labelled as “0”.
15. The method according to embodiment 13, further comprising:
in response to a regression model as the CNN model, labelling an output ground truth of each training sample, with the output ground truth indicating a high or low editing activity of sgRNA.
16. The method according to embodiment 15, wherein the output ground truth includes a phenotype score γ, which is calculated as follows:

$$phenotype score ( γ ) = \frac{{Log}_{2} sgRNA enrichment}{fold difference} .$$

17. The method according to any one of the preceding embodiments, wherein the one or more epigenetic features include but are not limited to: distance between transcription start site (TSS) and sgRNA target site, DNA methylation level, RNA expression level and chromosome accessibility.
18. The method according to embodiment 17, wherein the epigenetic features of DNA methylation level, RNA expression level and chromosome accessibility are quantified by whole-genome bisulfite sequencing data, RNA-seq data, and ATAC-seq data, respectively.
19. The method according to any one of the preceding embodiments, wherein the dataset is divided into a training set and a testing set at a ratio of 9:1.
20. The method according to any one of the preceding embodiments, further comprising: executing repeatedly the following process multiple times, namely dividing the dataset into a training set and a testing set, followed by training the CNN model using the input matrix and output ground truth of each sample in the training set, and determining the output accuracy of the trained model using the testing set, wherein the training set and testing set are randomly re-divided for each execution to ensure the stability of the model.
21. The method according to embodiment 10, wherein each of the five convolution layers uses 30 filters, and the convolution layers have a kernel size of 1, 2, 3, 4, and 5, respectively.
22. The method according to embodiment 10, wherein the three fully connected layers contain 80, 60 and 40 units, respectively.
23. The method according to embodiment 12, wherein the drop-out function has a drop-out rate of 0.4.
24. A method for predicting the gene editing activity of CRISPR/dCas sgRNA based on deep learning, comprising:
establishing a prediction model for predicting the gene editing activity of a CRISPR/dCas sgRNA based on a dataset comprising sgRNAs and editing activity data thereof, one or more epigenetic features, and a convolutional neural network model:
transforming the sgRNA sequence to be tested and related epigenetic features thereof into an input matrix suitable for the prediction model, and inputting it into the prediction model, so as to obtain a predicted value of activity of the sgRNA.
25. The method according to embodiment 24, wherein the one or more epigenetic features include: distance between transcription start site (TSS) and sgRNA target site, DNA methylation level, RNA expression level and chromosome accessibility.

The foregoing is an overview and thus simplifications, generalizations, and omissions of details are included if necessary: consequently, those skilled in the art will appreciate that the overview is illustrative only and is not intended to be in any way limiting. Other aspects, features and advantages of the methods, compositions and uses and/or other subject matters described herein will become apparent in the teachings set forth herein.

### BRIEF DESCRIPTION OF THE FIGURES

FIG. 1A illustrates a schematic diagram showing the dCas9 protein complex fused with a transcription repressor or transcription activator is targeted to a specific region of the genome under the guidance of a specific sgRNA to inhibit (CRISPRi) or activate (CRISPRa) the transcription of the target gene.

FIG. 1B illustrates a schematic diagram of the CRISPRoff/on editing system, which can increase or decrease DNA methylation and inhibitory histone modification by fusing the dCas9 protein with a methylase or demethylase, thereby achieving the purpose of reducing specific gene expression.

FIGS. 1C-F show the relationship between sgRNA-related epigenetic features and the editing efficiency of the sgRNA in the CRISPRoff system. FIG. 1C: distance between transcription start site (TSS) and sgRNA target site, FIG. 1D: chromosome accessibility, FIG. 1E: DNA methylation level, FIG. 1F: RNA expression level.

FIG. 2A illustrates the model diagram of Deep-epi, wherein a 40mer genomic DNA sequence and four epigenetic features are used as inputs for the model to construct a prediction model containing five convolution layers and three fully connected layers.

FIG. 2B shows the results of Spearman correlation coefficient obtained by selecting genomic sites with different lengths as model inputs.

FIGS. 2C and 2D show the ROC curves (a classification model) and Spearman correlation coefficient (a regression model) results predicted by the model obtained by training and testing the combination of sequence features and various epigenetic features.

FIGS. 3A and 3B show the ROC curves (a classification model) and Spearman correlation coefficients (a regression model) for the Deep-epi model and other models in the CRISPRoff testing set. The Gradient Boost, Random Forest, DeepHF, C-RNN, Deep-seq and Deep-epi models are indicated from left to right in FIG. 3B.

FIG. 3C shows the ROC curves and AUC values between the predicted and true values by the prediction of Deep-epi classification model and other models in four datasets.

FIG. 3D shows the Spearman correlation coefficients predicted by the Deep-epi regression model and other models in five datasets, with the Gradient Boost, Random Forest, C-RNN, DeepHF, Deep-seq and Deep-epi models indicated from left to right.

FIG. 3E shows the comparison results of the training time for the Deep-epi regression model and other models, with the C-RNN, DeepHF, Deep-seq and Deep-epi models indicated from left to right.

FIG. 3F shows the comparison results of MSE and KL divergence for the Deep-epi regression model and other models.

FIG. 4A shows the ROC curves and AUC values between the predicted and true values by the prediction of Deep-epi classification model in all five testing datasets.

FIG. 4B shows the Spearman correlation coefficient results predicted by the Deep-epi regression model in all five testing datasets. The Gradient Boost, Random Forest, C-RNN, DeepHF, Deep-seq and Deep-epi models are indicated from left to right.

FIG. 4C shows the Spearman correlation coefficient results predicted by the Deep-epi regression model and other models in different types of cell lines.

FIG. 4D shows a scatter plot of the correlation between the predicted and true values from Deep-epi in the four exemplified genes.

FIG. 5A shows the Spearman correlation coefficient results for the CRISPRa training set by selecting genomic sites with different lengths as inputs.

FIG. 5B shows the ROC curves (a classification model) and AUC values predicted by the model obtained by combining sequence features with various epigenetic features for training and testing the CRISPRa dataset.

FIG. 5C shows the ROC curves (a classification model) and AUC values predicted by Deep-epi and other models in the CRISPRa dataset.

FIG. 5D shows the Spearman correlation coefficient results predicted by the model obtained by combining sequence features with various epigenetic features for training and validating the CRISPRa dataset.

FIG. 5E shows the Spearman correlation coefficient results predicted by Deep-epi and other models in the CRISPRa dataset (hCRISPRav2).

FIG. 5F shows the ROC curves and AUC values between the predicted and true values predicted by the Deep-epi classification model in one CRISPRa testing dataset.

FIG. 5G shows the Spearman correlation coefficient results predicted by the Deep-epi regression model in one CRISPRa testing dataset (CRISPRa_intergrate). The models in FIGS. 5E-G are all arranged in the following order: from left to right, Gradient Boost, Random Forest, DeepHF, C-RNN, Deep-seq and Deep-epi models.

FIG. 6A shows a histogram of the Tree SHAP results of the Deep-epi model on multiple features in the CRISPRoff training set.

FIG. 6B shows a histogram of the Tree SHAP results of the Deep-epi model on multiple features in the CRISPRa training set.

FIG. 6C shows the global SHAP values of the Deep-epi model in the CRISPRoff training set.

FIG. 6D shows the global SHAP values of the Deep-epi model in the CRISPRa training set.

### DETAILED DESCRIPTION

Organisms, especially chromosomes composed of histones and DNA, are extremely complex biological systems that contain many epigenetic features. Therefore, when establishing a tool for predicting the editing efficiency of sgRNA, it may be closer to the in vivo environment of organisms to include more epigenetic features, especially for an epigenetic editing tool. The inventors first dissected the relationship between the editing efficiency of the CRISPR/dCas system and epigenetic features, and explored the features that play an important role in the epigenetic editing system, including sequence features and epigenetic features. Subsequently, an epigenetic editing prediction tool (named as Deep-epi herein) was built based on the deep learning model.

As used herein, the term “epigenetic” refers to heritable alterations in gene expression without changing the nucleotide sequence of a gene, including DNA methylation, genomic imprinting, maternal effect, gene silencing, nucleolar dominance, dormant transposon activation and RNA editing. Epigenetics is a complex process influenced by a range of cellular factors. In CRISPR-based epigenetic editing systems (such as CRISPRoff), gene expression is targeted through epigenetic modifications, in which the epigenetic modifications are carried out by DNA methylation, DNA demethylation, histone acetylation or methylation and others at regulatory elements (e.g., a promoter, enhancer, or transcription start site) of a target gene, thereby directing gene transcription or gene silencing/repression. For example, methylating DNA in a region that regulates transcriptional activity will alter gene expression but not DNA sequence. Transcriptional regulation through epigenetic modifications (e.g., DNA methylation) allows targeted regulation of gene expression without affecting the expression of other gene products. Fusion of the dCas protein without cleavage activity with transcription regulators (such as DNA methylases and demethylases) provides a powerful tool for regulating transcription levels, greatly promoting epigenetic research [Silvana Konermann et al., Genome-scale transcriptional activation by an engineered CRISPR-Cas9 complex, Nature. 2015 Jan. 29: 517 (7536): 583-8].

The term “dCas” herein refers to the RNA-guided nuclease in the CRISPR/dCas gene editing system, which has been modified to have no nickase activity, and thus without the ability to create nicks on any strand of DNA. Nucleases without nickase activity may be, for example, dCas9, dSpGCas9, dSpYRCas9 nucleases, dLbCpf1, dAsCpf1 and denAsCpf1 nucleases.

As used herein, the term “target site” refers to the region on DNA where the protospacer sequence is located.

As used herein, the term “guide RNA” or “gRNA” refers to an RNA sequence that comprises a guide sequence and, optionally, a tracrRNA. Common guide RNAs are composed of crRNA and tracrRNA sequences that form complexes through partial complementarity, where the sequence contained in the crRNA is sufficiently complementary to the target sequence to hybridize, and direct the CRISPR complex to a specific binding target sequence. The term also includes a single guide RNA (sgRNA), which contains features of both crRNA and tracrRNA. Typically, the guide sequence of a gRNA is complementary to the target nucleic acid sequence and is responsible for initially guiding RNA/target base pairing. Preferably, the guide sequence of the gRNA does not tolerate mismatches. In the present invention, the terms “gRNA” and “sgRNA” are used interchangeably.

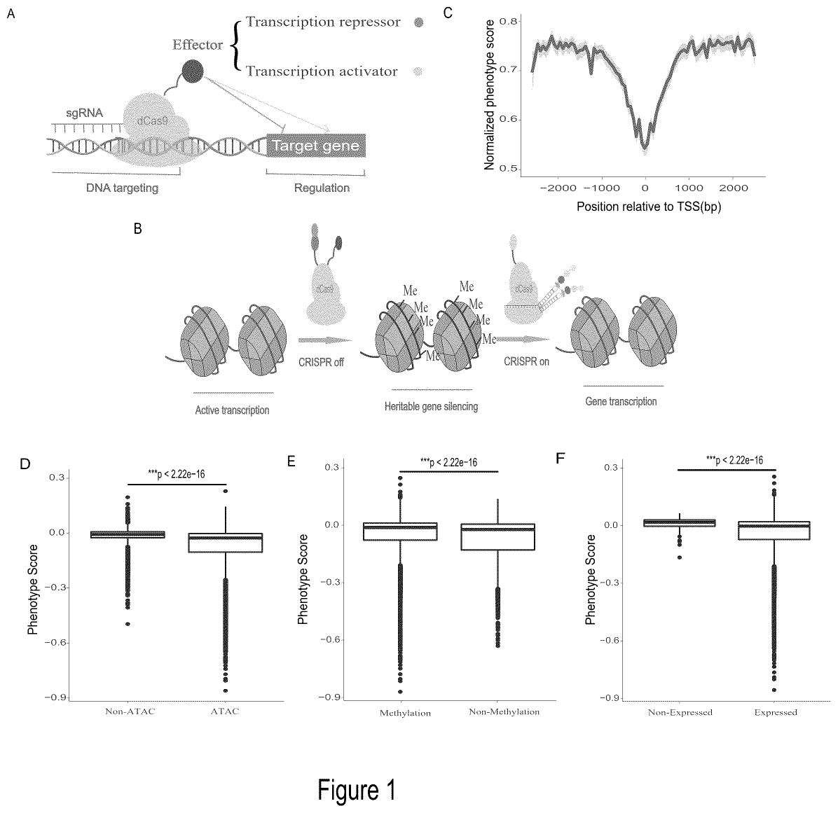

As used herein, the terms “CRISPRoff”, “CRISPRi”, “CRISPRon” and “CRISPRa” all refer to epigenetics-based CRISPR/dCas gene editing systems, in which a dCas protein without cleavage activity is fused with a transcription regulator (such as a DNA methylase, demethylase, transcription repressor and transcription activator) to regulate the transcription level of a target gene. Among them, CRISPRi is a process that fuses a dCas protein with a transcription repressor such as KRAB, CRISPRoff is a process in which a methyltransferase, such as DNMT3A/3L, is further fused on the basis of CRISPRi, CRISPRa is a process that fuses a dCas protein with a transcription activator such as VP64 or p65-AD or Rta, and CRISPRon is a further fusion of demethyltransferase, such as TET1, on the basis of CRISPRa. Schematic diagrams of the CRISPRoff and CRISPRon systems are shown in FIGS. 1A and 1B.

As used herein, the dCas protein in the CRISPR/dCas gene editing system has binding affinity to a polynucleotide sequence motif in the target nucleic acid, and the sequence motif is usually known as a “protospacer adjacent motif” or “PAM”. Preferably, the sequence motif contains 3 or more contiguous nucleotide residues. PAM is located on the target strand, adjacent to the protospacer sequence.

As used herein, the term “editing efficiency” refers to the proportion of target sequences that are epigenetically modified with the intention of altering their expression. In the present invention, editing efficiency mainly refers to the editing efficiency in epigenetics, where the nucleotide sequence of the target sequence remains unchanged but its expression undergoes heritable changes.

The contents of all references, patents, and published patent applications cited in this application are incorporated herein by reference in their entirety. Furthermore, any section headings used herein are for organizational purposes only and are not to be construed as limiting the subject matter described.

I. Epigenetic Features for Improving the Prediction of sgRNA Editing Efficiency

In the present disclosure, four types of epigenetic features that play important roles in the development of epigenetics were identified for the epigenetics-based CRISPR/dCas gene editing system, namely: distance between transcription start site (TSS) and sgRNA target site, DNA methylation level, RNA expression level and chromosome accessibility. The incorporation of these epigenetic features as input in modeling significantly improved the prediction efficiency of gene editing of sgRNA.

For features of DNA methylation level, RNA expression level and chromosome accessibility, whole-genome bisulfite sequencing (WGBS) data, RNA-seq data, and ATAC-seq data were used to quantitatively represent these features, respectively. Subsequently, the relationship between these epigenetic features and editing efficiency in the CRISPRoff system was explored. As shown in FIGS. 1C-F, all four of these features significantly affected the editing efficiency in the CRISPRoff system.

II. Prediction Models for Editing Systems that Inhibit or Activate Gene Expression

Editing systems that inhibit gene expression include the CRISPRoff epigenetic modification editing system and the CRISPRi system. In some exemplary embodiments, the training dataset selected for use was a CRISPRoff_tiling dataset, which contained 116,000 unique sgRNAs, with 111,638 sgRNAs left after removal of non-targeting sequences.

In some predictions for editing systems (such as CRISPRa/on) that activate gene expression, the training dataset selected was a hCRISPRav2 dataset, which contained 198,757 sgRNAs.

The epigenetic editing efficiency was expressed as a phenotype score γ. The γ value is a function of the enrichment degree before and after the sgRNA experiment, and the greater the absolute value of the γ is, the higher the sgRNA activity is. Since CRISPRoff or CRISPRi has an inhibitory effect on gene expression, a more negative γ value in this type of dataset represents a better effect of gene editing: on the contrary, in the CRISPRa/on dataset, a more positive γ value represents a better effect of gene editing. The dataset was randomly divided into a testing set of 10%, and the remaining 90% is used for model training.

### Input to the Model:

1. Target sequence features. Preferably, a target sequence with a length of 40mer, containing an upstream sequence of 9 base-pairs (bp), a protospacer (i.e., an sgRNA sequence feature) of 20 base-pairs (bp), a PAM sequence of 3 base-pairs (bp) and a downstream sequence of 8 base-pairs (bp), is selected as the input to the model.
2. One or more of the following epigenetic features: distance between transcription start site (TSS) and sgRNA target site, DNA methylation level, RNA expression level and chromosome accessibility, of which whole-genome bisulfite sequencing (WGBS) data were used to quantify the DNA methylation level, RNA-seq data were used to quantify the RNA expression level, and ATAC-seq data were used to quantify the chromosome accessibility level.

One-hot encoding is used to convert the above features into a binary matrix (X), and the phenotype score is the corresponding numerical value (Y): the input layer uses a linear activation function to better train the data.

### Convolutional Neural Network Model Building:

1. Five convolution layers extract features from the input matrix in parallel, using 30 1-nt filters, 30 2-nt filters, 30 3-nt filters, 30 4-nt filters, and 30 5-nt filters, respectively: the Relu activation function is used:
2. Three fully connected layers, containing 80, 60 and 40 units, respectively:
3. Optionally, a drop-out regularization function can be added behind the convolution layers and fully connected layers to avoid overfitting the training set.

For simplicity, the prediction model obtained by training with the above method is referred to as Deep-epi model herein. The convolution layers of Deep-epi consist of five filters with different sizes to extract sufficient information from the sgRNA sequences and their surrounding sequences in an unsupervised manner.

### III. Evaluation Method for Editing Efficiency Prediction Results

### 1. Comparison Between Deep-Epi Classification Model and Other Prediction Models

In the classification model, 111,638 sgRNAs in the CRISPRoff_tiling database are classified according to editing efficiency, with those with significant editing effects labelled as “1” and the remaining ones labelled as “0”. The dataset is then randomly divided into a testing set of 10%, and the remaining 90% is used for model training. A variety of models known in the art can be employed to compare with the Deep-epi classification model, including but not limited to: Random Forest, Gradient Boost, Deep HF and C-RNN.

Random Forest: it refers to a classifier using multiple decision trees to train and predict samples. The classifier was first proposed by Leo Breiman and Adele Cutler. Random Forest is composed of a number of decision trees with no correlation between different decision trees. When performing a classification task, whenever a new input sample comes in, each decision tree in the forest is allowed to judge and classify individually, in this way each decision tree will obtain its own classification result, and the result that gets the most out of these classification results from all decision trees will be taken as the final result by the Random Forest.

Gradient Boost: Boosting is a method of obtaining one strong learner by the following steps, i.e., combining a group of weak learners, with low complexity, low training cost and less susceptibility to overfitting, to establish N models (classifications), and in each classification, trying to increase the weight of the data that are misclassified in the previous classification a little bit before proceeding with next classification. Gradient Boosting is a boosting method, and also an ensemble learning algorithm and machine learning technique commonly used for regression and classification problems, which generates prediction models by integration of a set of weak prediction models (usually decision trees). The main concept is that each time a model is built, it will follow the direction of a gradient descent in the loss function of the previously built model, that is, these models are generated by optimizing the loss function.

DeepHF: it is based on the RNN (Recurrent Neural Network) framework in deep learning, using the embedding encoding pattern. This model combines both sequence and RNA secondary structure features, first using RNN to train sequence features, and then adding secondary features for full connection after training. For specific methods, see Daqi Wang et al., Optimized CRISPR guide RNA design for two high-fidelity Cas9 variants by deep learning, Nat Commun. 2019 Sep. 19: 10 (1): 4284.doi: 10.1038/s41467-019-12281-8.

C-RNN (Convolutional Recurrent Neural Network): The RNN network will memorize the previous information and apply it to the calculation of the current output, that is, the nodes between the hidden layers are no longer connectionless but connected, and the input of the hidden layers includes not only the output of the input layer but also the output of the hidden layer at the previous moment. C-RNN is often used to solve image-based sequence recognition problems.

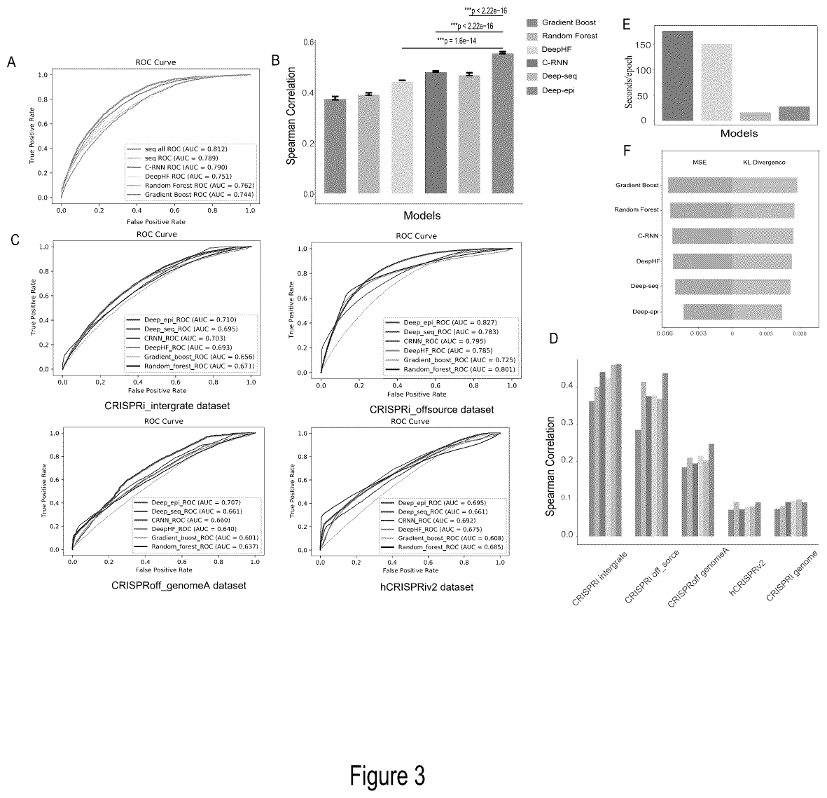

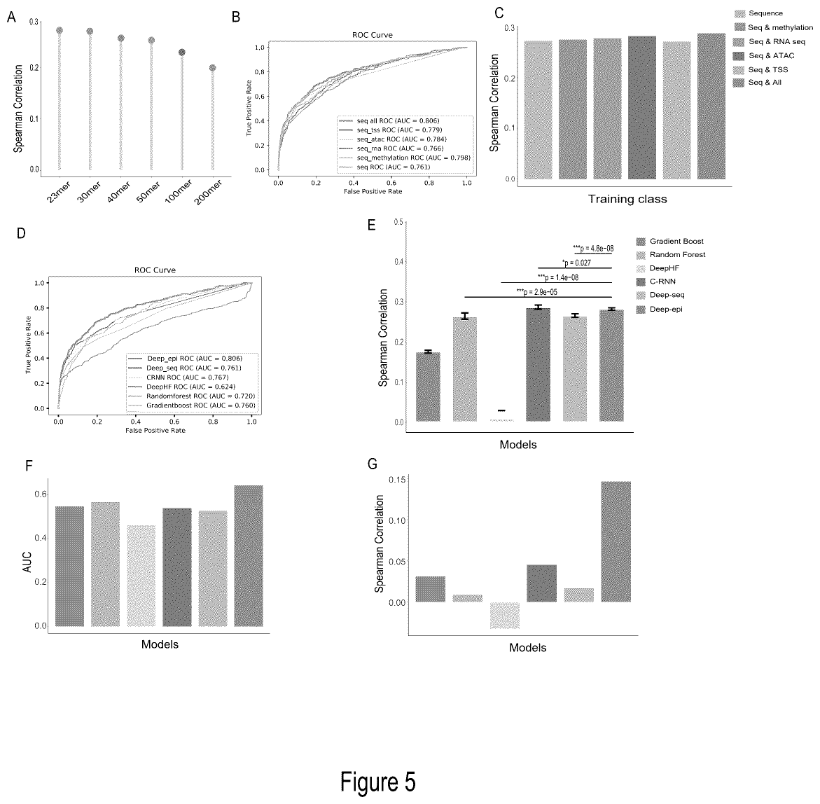

Comparison Criteria: Area under the curve (AUC) of ROC curve (receiver operating characteristic curve). The ROC curve can be used to describe a process by which classifier performance varies with changes in the classifier threshold. For the ROC curve, an important feature is the area under the curve, with an area of 0.5 indicating random classification and a recognition ability of 0; the closer the area is to 1, the higher the recognition ability is, and an area equal to 1 is considered as complete recognition. As shown in FIGS. 3A and 5D, compared with the four classical models, the Deep-epi model (including sequence features and all four epigenetic features) disclosed in the present invention has the greatest AUC value, significantly higher than that of all other models. The AUC value of the control model Deep-seq, which contains no epigenetic features but only sequence features, is comparable to the resultant AUC value of C-RNN.

### 2. Comparison of Prediction Effects of Deep-Epi Classification Model in Other Datasets

In order to evaluate the stability and generalization of the model, comparisons were also conducted in 5 datasets from different sources: CRISPRoff_genomeA, CRISPRi_intergrate, CRISPRi_genome, CRISPRi_off_score and hCRISPRiV2. These 5 experimental datasets are derived from different cell lines and correspond to CRISPRi experimental results. Corresponding to CRISPRa, the CRISPRa_intergrate dataset is used. As shown in FIGS. 3C and 3D, in all tested datasets, the Deep-epi model has the greatest AUC value or Spearman correlation value compared with other models, indicating that the model has better stability and generalization, moreover the model is equally applicable to both CRISPRi editing systems and CRISPRa editing systems.

### 3. Deep-Epi Regression Model

In the Deep-epi regression model, the editing efficiency value γ corresponding to each sample in the training set is normalized and then input into the model. The Deep-epi regression model is compared with known models.

### Comparison Criteria:

### (A) Spearman's Rank Correlation Coefficient

This coefficient is a non-parametric index to measure the dependence between two variables. It uses a monotone equation to evaluate the correlation between two statistical variables. If there are no duplicate values in the data, and when the two variables are completely monotonically correlated, the Spearman correlation coefficient is +1 or −1.

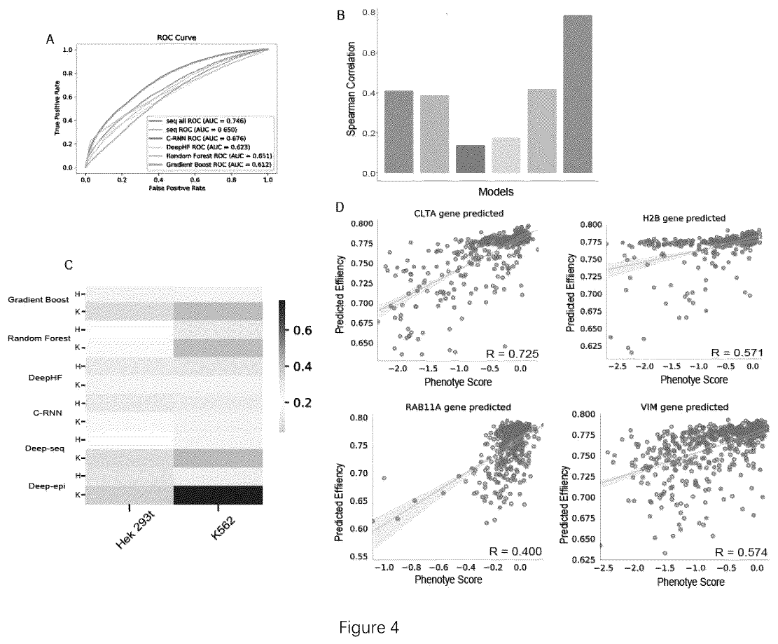

As shown in FIGS. 4A and 4D, except for CRISPRoff_genomeA, the Deep-epi model has the highest coefficients in CRISPRoff_tiling datasets.

### (B) Mean Squared Error

The mean squared error (MSE) is a measure that reflects the degree of difference between the observed and predicted values. As shown in FIG. 4C, the MSE value of the Deep-epi model is significantly lower than other models.

### (C) Kullback-Leibler Divergence

KL divergence, or relative entropy, measures the difference between two probability distributions in the same event space, with the two probability distributions representing the true distribution of data and the theoretical or model distribution of data, respectively.

As shown in FIG. 4C, the KL divergence of the Deep-epi model is significantly lower than other models.

### IV. Importance of Different Epigenetic Features in Model Prediction

In evaluating the importance of different features, the SHapley Additive explanations (SHAP) method is used, in which SHAP constructs an additive explanatory model inspired by cooperative game theory, considering all features as “contributors”. For each prediction sample, the model generates a predicted value, and the SHAP value is the numerical value assigned to each feature in that sample. The greatest advantage of SHAP values is that SHAP can reflect the influence of the features in each sample, and also show the positive and negative effects of the influence. Scott M. Lundberg et al. also proposed TreeSHAP to provide a local explanation of the tree model, and TreeSHAP does not require sampling, but calculates SHAP values by analyzing nodes in the tree model (Scott M. Lundberg et al., From local explanations to global understanding with explainable AI for trees.2020).

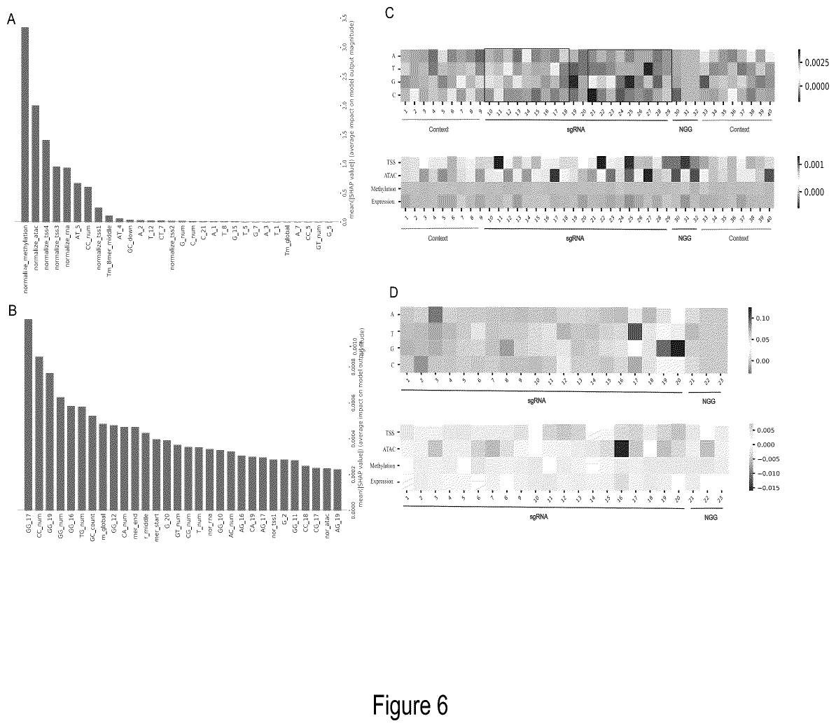

The greater the absolute value of the SHAP value, the heavier the influence of that feature. As shown in FIG. 6A, the four features of methylation, ATAC, TSS, and RNA expression were ranked at the top four, validating the importance of these features.

### V. Advantageous Effects

Through a comprehensive and systematic analysis of various features affecting gene editing of sgRNA, the inventors have constructed a deep learning model suitable for predicting gene editing efficiency in the CRISPR/dCas epigenetic editing system, providing a convenient tool for selecting sgRNAs for target genes. This model is not only applicable to CRISPR/dCas epigenetic editing systems, but can also be used for other types of CRISPR/Cas editing systems.

The present disclosure provides a method for selecting sgRNAs for any given desired genomic target region. In various aspects, the deep learning model disclosed herein may involve considering one or more sequence features, environmental features surrounding the sequence, epigenetic features, including but not limited to target nucleotide sequence (such as sgRNA binding site), target genomic location, transcriptional status of target genomic location, cell type, accessibility of chromosomal region, and the like. The present invention has achieved at least the following beneficial effects:
1. A convolutional neural network is used as the basic framework, and one or more epigenetic features are introduced as inputs on the basis of sequence features, so as to reinforce the simulation of the in vivo environment of organisms and improve the accuracy of prediction.
2. The model structure has high prediction accuracy in a wide range of datasets and has good generalization performance.
3. The time complexity is low, the iteration time is short, and the practicality is high.
4. For the first time, a prediction tool in the CRISPR/dCas epigenetic editing system is provided.

### EXAMPLES

In order to facilitate understanding and implementation, the present invention will be further described below in conjunction with examples, and it should be understood that the examples described here are only for the purpose of illustrating and explaining the present invention, and are not intended to be limiting.

### 1. Datasets

Epigenetic editing systems can be divided into two categories according to their functions, namely, editing systems that inhibit gene expression and those that activate gene expression. Two prediction models were designed for these two categories of editing systems with differential functions, respectively. For the prediction tool for inhibiting gene expression, the training dataset selected was CRISPRoff_tiling data [from James K Nuñez et al., Genome-wide programmable transcriptional memory by CRISPR-based epigenome editing, Cell. 2021 Apr. 29: 184 (9): 2503-2519.e17]. The dataset is obtained from CRISPRoff screening experiments in HEK293T cells, consists of 520 genes, and for each gene, the entire sequence spanning 2.5 kb upstream and downstream of the transcription start site (TSS) with a protospacer adjacent motif (PAM) therein was selected, containing a total of 116,000 unique sgRNAs. 111,638 sgRNAs remained after removal of non-targeting sequences. The dataset was used to build a benchmark model and adjust parameters, and explore the important features of the model. Here, the epigenetic editing efficiency of CRISPRoff was expressed as a phenotype score (γ), which was calculated as follows:

$$phenotype score ( γ ) = \frac{{Log}_{2} sgRNA enrichment}{fold difference}$$

In the experiment, the dataset was randomly divided into 10 parts, with a ratio of 9:1 between the training and testing sets. The training set was used to train the model, a process in which the testing set was not involved, and the trained model was tested with the testing set. This process was repeated 20 times to ensure the model is stable and avoid instability incurred by data deviation. In order to evaluate the stability and generalization of the model, the prediction performance of the model in 5 datasets from different sources was also evaluated: CRISPRoff_genomeA, CRISPRi_intergrate, CRISPRi_genome, CRISPRi_CRISPRoffsource and hCRISPRiV2 [Table 1].

For the prediction tool for activating gene expression, the dataset used for model training was hCRISPRav2, which was derived from Max A Horlbeck et al., Compact and highly active next-generation libraries for CRISPR-mediated gene repression and activation, Elife. 2016 Sep. 23; 5: e19760. doi: 10.7554/eLife. 19760. Similarly, the dataset was randomly divided into 10 parts, with a ratio of 9:1 between the training and testing sets, and the model was constructed in the same manner as described above.

TABLE 1
Information of the datasets

| | | | Number | |
|---|---|---|---|---|
| Datasets | Types | Cell lines | of RNAs | Literature sources |
| CRISPRoff tiling | CRISPRoff | Hek293t | 111,683 | JAMES K NUÑEZ et |
| | | | | al., supra |
| CRISPRoff_genomeA | CRISPRoff | Hek293t | 20,222 | JAMES K NUÑEZ et |
| | | | | al., supra |
| CRISPRi_CRISPRoffs | CRISPRi | K562 | 111,284 | JAMES K NUÑEZ et |
| | | | | al., supra |
| hCRISPRiv2 | CRISPRi | K562 | 199,524 | Max A Horlbeck et al., |
| | | | | supra |
| CRISPRi_genome | CRISPRi | K562 | 107,576 | Luke A Gilbert et al., |
| | | | | supra |
| CRISPRi_intergrate | CRISPRi | K562 | 18,080 | Max A Horlbeck et al., |
| | | | | supra |
| hCRISPRav2 | CRISPRa | K562 | 198,757 | Max A Horlbeck et al., |
| | | | | supra |
| CRISPRa_intergrate | CRISPRa | K562 | 2780 | Max A Horlbeck et al., |
| | | | | supra |

### 2. Correlation Between Epigenetic Features and Editing Results

In this work, four categories of epigenetic features that play important roles in the development of epigenetics were selected, which were respectively as follows: distance between transcription start site (TSS) and sgRNA target site, DNA methylation level, RNA expression level and chromosome accessibility. Whole-genome bisulfite sequencing (WGBS) data, RNA-seq data and ATAC-seq (Assay for Transposase Accessible Chromatin using sequencing) data were used to quantify these features, among which, WGBS data corresponded to the DNA methylation level, RNA-seq data corresponded to the RNA expression level, and ATAC-seq data corresponded to the chromosome accessibility (Table 2). Subsequently, an attempt was made to explore the relationship between these epigenetic features and CRISPRoff editing efficiency.

TABLE 2
Sources of epigenetic data

| | Data | | |
|---|---|---|---|
| Cell lines | types | Sources | Links |
| Hek293t | RNAseq | GSE152177 | https://www.ncbi.nlm.nih.gov/geo/query/acc.cgi?acc=GSE152177 |
| Hek293t | ATAC | GSE152177 | https://www.ncbi.nlm.nih.gov/geo/query/acc.cgi?acc=GSE152127 |
| Hek293t | Methylation | GSM720355 | https://www.ncbi.nlm.nih.gov/geo/query/acc.cgi?acc=GSM720355 |
| K562 | RNAseq | GSE114071 | https://www.ncbi.nlm.nih.gov/geo/query/acc.cgi?acc=GSE114071 |
| K562 | ATAC | ATACdb | http://www.licpathway.net/ATACdb/search/search_sample_result.php?get_sample_id=101 |
| | | Sample_0101 | |
| K562 | Methylation | GSM1589167 | https://www.ncbi.nlm.nih.gov/geo/query/acc.cgi?acc=GSM1589167 |

Consistent with previous studies, highly active sgRNAs were concentrated within a narrow window around the TSS (FIG. 1C). In addition, it was found that the editing efficiency of CRISRPoff was related to chromosome accessibility, and the RNA expression profile was also related to editing efficiency (FIGS. 1D, 1F). However, sites with DNA methylation exhibited lower editing activity relative to unmethylated sites, which could be explained by the fact that high DNA methylation levels reduce chromosome activity, making it difficult for dCas complexes to bind (FIG. 1E).

### 3. Construction of Deep Learning Models

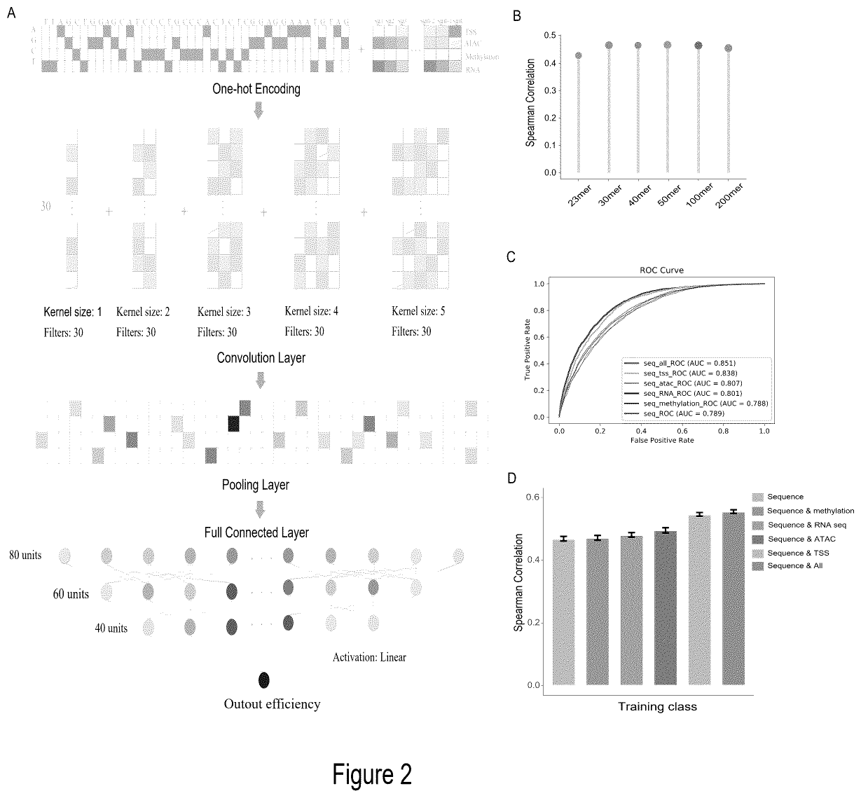

Based on the previous research results, an attempt was made to develop a model to accurately predict the editing activity of CRISPRoff. A prediction model containing five convolution layers and three fully connected layers, entitled Deep-epi, was built using the convolutional neural network (CNN) in deep learning. It has been shown that the editing efficiency of CRISPR/Cas systems correlates with the environment around the sgRNA binding site. Therefore, we first designed genomic sites of different lengths for study, including 23mer, 30mer, 40mer, 50mer, 100mer and 200mer [FIG. 2B, FIG. 5A]. Although the results obtained at 30mer were similar to those obtained at 40mer, 40mer was chosen as the input to the model because it allowed for providing more sequence and epigenetic information. The 40mer length includes an upstream sequence of 9 base-pairs (bp), a protospacer of 20 base-pairs (bp), a PAM sequence of 3 base-pairs (bp) and a downstream sequence of 8 base-pairs (bp).

As shown in the model diagram of FIG. 2A, the DNA sequence of 40mer was converted into a binary matrix (X1) using one-hot encoding, where the rows of the matrix X1 represented the position of each nucleotide in the sequence, and the columns represented 4 base types at each nucleotide position: The numerical values of four epigenetic features corresponding to each base of 40mer were normalized and transformed into a continuous variable matrix (X2), where the rows of the matrix X2 represented the position of each nucleotide in the sequence, and the columns represented the expression levels of the four epigenetic features at each nucleotide position. The matrix X1 and matrix X2 were concatenated together as an input matrix (X) of the deep learning model, with the phenotype score being the corresponding numerical value (Y).

A total of 150 filters of different sizes (30 1-nt filters, 30 2-nt filters, 30 3-nt filters, 30 4-nt filters and 30 5-nt filters) were used in the convolution layers, and these filters extracted features from the input matrix in parallel. Here, the rectified linear unit (Relu) activation function was used in the convolution layers. Subsequently, the outputs of the convolution layers were concatenated together as inputs to the fully connected layers, and the three fully connected layers contained 80, 60 and 40 units, respectively. To avoid overfitting during the training process, a drop out function (Drop out=0.4) was added after the convolution layers and fully connected layers. For the input layer, a linear activation function (linear) was used to better train the data.

Previous results have shown that epigenetic features can affect the efficiency of CRISPRoff. In order to explore the influence of epigenetic features on the prediction ability of models, multiple prediction models were established. They were divided into two categories of prediction models, regression models and classification models, and each category included six models with different epigenetic features and different prediction modes: (1) only the sequence features (named Deep-seq), with the addition of (2) DNA methylation feature, (3) RNA seq feature, (4) ATAC seq feature, (5) TSS feature, and (6) DNA methylation+RNA seq+ATAC seq+TSS. After training and verification according to the methods mentioned above, the results of both categories of models showed that the model with all features combined together performed the best in prediction results, that is, the model (6) (FIGS. 2C, 3A and FIGS. 5B-5C). Also, the addition of epigenetic features one by one showed a significant improvement compared to sequence features, indicating that the four selected epigenetic features played an important role in predicting editing efficiency. Therefore, the final prediction model, known as Deep-epi, was established by combining sequence features with four epigenetic features.

4. Comparison with Other Models

### 4.1 Classical Models

111,638 sgRNAs in the CRISPRoff_tiling database were classified according to editing efficiency, with those with significant editing effects labelled as “1” and the remaining ones labelled as “0”. According to the experimental approach mentioned above, the dataset was randomly divided into a testing set of 10%, and the remaining 90% was used for model training. The CNN-based classification models were separately incorporated with epigenetic features to undergo unsupervised training, and the trained models were compared with other prediction tools. In order to better evaluate the prediction ability of Deep-epi, four different types of prediction models were selected for comparison, namely Random Forest, Gradient Boost, DeepHF, and C-RNN.

The reasons for choosing these types of models were as follows: (1) Random Forest and Gradient Boost are two powerful machine learning algorithms, which are popular in many data prediction applications. (2) DeepHF is based on the RNN framework in deep learning, and the data encoding pattern is the embedding encoding pattern. (3) The model framework of C-RNN contains a CNN framework that is consistent with Deep-epi and, in addition, the model is incorporated with the BGRU module. The comparison criterion used was the value of the area under the receiver operating characteristic curve (AUC). The greater the value, the better the prediction effect. A comparison of the prediction results from the testing set in FIG. 3A showed that Deep-epi had an AUC value of 0.812, which was superior to other models. In FIG. 5D, the Deep-epi model for CRISPRa also yielded the best results.

### 4.2 Regression Model

Meanwhile, a regression prediction model was provided. In the training of this model, the editing efficiency value γ corresponding to each sample in the training set was normalized and then input into the model. Consistent with the above mentioned method, the Deep-epi regression model was compared with the other four models, and Spearman's rank correlation coefficient was used as a criterion for evaluating these models. Similarly, it was concluded that Deep-epi performed better than other models (FIG. 3B).

In addition, the training time of a deep learning model is also an important factor in model performance evaluation, as a model with a short training time is more likely to be applied to datasets with large volumes. Therefore, the training times of four deep learning models, including Deep-epi, Deep-seq, DeepHF, and C-RNN, were compared, and the time required for each iteration was taken as the criterion (FIG. 3E). The results showed that the time required for Deep-seq was slightly shorter than Deep-epi, but previous results showed that the prediction performance of Deep-epi was superior to that of Deep-seq, indicating that Deep-epi had a better prediction performance and could save a lot of computer resources. In order to compare these models more comprehensively, two indices, Mean Squared Error (MSE) and Kullback-Leibler Divergence (KL divergence) were used to compare the predicted results of the models with the true results on the testing set, and it could be concluded that the difference between the predicted value and the true value was smallest for Deep-epi (FIG. 3F).

### 5. Generalization on Other Datasets

The action mechanism of CRISPRoff is similar to that of CRISPRi, that is, the inhibition of gene transcription occurs both through fusing the dCas protein with effectors. Therefore, it is speculated that the model generated by CRISPRoff training can also be used to predict the editing efficiency of CRISPRi. Here, five experimental datasets from different cell lines of two editing systems were selected for testing the generalization ability (Table 1). The classification model and regression model of Deep-epi were separately used for prediction in five datasets, and the ROC-AUC values or Spearman correlation between the predicted and true values were calculated (FIG. 3C, 3D). According to the results, Deep-epi was essentially superior to other models in the comparison of classification and regression models. Among them, the overall prediction effect in CRISPRoff_genomeA was relatively low, and the reason may be that the dataset was derived from a CRISPRoff dual sgRNA experiment, which was quite different from the Deep-epi training set in experimental approaches. Similarly, models generated from CRISPRi training could be tested for generalization ability on CRISPRa_intergrate.

The dataset of four genes, VIM, CLTA, H2B and RAB11A, was derived from the CRISPRoff tiling screening experiment, and the results shown here were scatter plots between the predicted and true values from the regression model, so as to reveal the relationship between the predicted and true values of Deep-epi more intuitively. As a result, it could be concluded that there was a strong correlation between the predicted and true values of Deep-epi in the four genes (FIG. 4D). 6 Analysis of important features by SHAP

Next, attention was turned to the importance of different features in deep learning models. SHapley Additive explanations (SHAP) is a game-theoretic method that can be used to explain the output from any machine learning model. It links optimal credit allocation to the partial explanation of the classical Shapley value and its related extension using the game theory, which has been applied to the feature interpretation of several CRISPR system prediction tools.

Firstly, a brief analysis was conducted on the different types of nucleotides and the number of nucleotides in the CRISPRoff_tiling dataset. Subsequently, Tree SHAP, which combines the SHAP and XGBoost algorithms, was used to implement our feature analysis. Here, in order to identify features that contribute more to editing efficiency, 486 features were extracted from four different aspects, including position-related sequence features, number of different types of nucleotides, secondary structure features of sgRNA and epigenetic features. The results of Tree SHAP were displayed in a histogram (maximum display value for features was 30) (FIG. 6A). Basically consistent with the results obtained above, epigenetic features such as DNA methylation, TSS, RNA expression, and ATAC were all ranked at the top of the feature importance scale, indicating that these epigenetic features contributed greatly to the accuracy of the model. Studies have identified multiple sequence-related features that affect the prediction performance of CRISPR/Cas9. In this study, it was found that the Tm values of PAM-surrounding sequences had a favorable effect on the model prediction. In addition, GC/GG/CG motifs played a positive role in model prediction, while AG/TA/AC/GA did the opposite. Specifically, the content of GC nucleotides played an important role in the model, consistent with previous research results, probably because sgRNAs with high GC content bound more robustly to the target sequence.

Immediately afterwards, the DeepSHAP interpreter, a high-speed approximation algorithm, was used to interpret the global SHAP values of the Deep-epi deep learning model (FIG. 6B). The contribution of each position-dependent nucleotide to sgRNA activity was calculated based on the average of 100 randomly selected sgRNAs from the training data. Similar to the results of TreeSHAP, the epigenetic features TSS and ATAC exhibited a strong promoting effect around the PAM sequence of the target sequence. The nonsignificant feature importance of DNA methylation and gene expression may be due to the presence of too many 0 values in the dataset.

### DISCUSSION

CRISPR-related editing systems have been widely used in basic research of life science. The design and selection of sgRNAs with excellent performance is currently an important goal in research fields related to gene editing. However, most of the currently available prediction algorithms are designed based on CRISPR/Cas gene editing systems. According to the knowledge of the inventors, Deep-epi is the first algorithm designed to predict the efficiency of CRISPR/dCas epigenetic editing systems by using deep learning models. Deep-epi utilizes the CRISPRoff dataset as a training set to establish a benchmark model, and then builds a prediction model for CRISPRa on this basis. According to different experimental criteria, the prediction models for two categories of editing systems both contain two types of models, i.e., classification and regression models. Deep-epi demonstrated excellent prediction capabilities compared with four different types of prediction tools on five independent datasets. The convolution layers of Deep-epi consist of multiple filters with different sizes to extract sufficient information from the sgRNA sequences and their surrounding sequences in an unsupervised manner. A drop-out regularization function is applied in the model to avoid overfitting the training set. In addition, we explored the important features involved in the functioning of the CRISPR/dCas epigenetic editing system.

In the past, numerous innovative efforts have advanced the understanding in the field of epigenetics by affecting biological functions. With the development of CRISPR-related systems, it has become possible to detect epigenetics-related functions in a site-directed and high-throughput manner. At the same time, the methods for treating genetic diseases through the precise control of biological functions have been expanded, making it possible to improve human related health conditions. Establishing sgRNA prediction models for CRISPR/dCas systems will greatly accelerate epigenetic research. CNN is one of the most typical frameworks in deep learning and excels in natural language processing. Using the CNN framework, Deep-epi has shown good prediction results on all datasets of CRISPR/dCas-related epigenetic editing systems. Moreover, models built with the CRISPRoff dataset as the training set can also be used for CRISPRi sgRNA prediction. Although there are many functional similarities between CRISPRoff and CRISPRi, the general principles regulating the activity of genomic sites in CRISPRoff remain largely unclear due to the lack of CRISPRoff-related experiments currently. Therefore, we have explored the sequence and epigenetic features of sgRNAs, which will be beneficial to improve the prediction algorithm with a view to better designing sgRNAs with high activity.

Overall, the present disclosure focuses on demonstrating Deep-epi as the first prediction model for epigenetic editing systems developed based on a deep learning model. By exploring the relationship between four representative epigenetic features and editing efficiency, Deep-epi's predictions are brought closer to an organism's environment. Looking ahead, it is of great significance to facilitate epigenetic research by designing active sgRNAs for CRISPR/dCas-related epigenetic editing systems.

Those skilled in the art will further realize that the invention can be implemented in other specific forms without departing from the spirit or central features thereof. In that the foregoing description of the present invention discloses only exemplary embodiments thereof, it is to be understood that other variations are recognized as being within the scope of the present invention. Accordingly, the present invention is not limited to the particular embodiments which have been described in detail herein. Rather, reference should be made to the appended claims as indicative of the scope and content of the present invention.

1. A model training method for predicting the editing activity of a sgRNA in a CRISPR/dCas gene editing system, comprising:
constructing or acquiring a sample dataset comprising sgRNAs and editing activity data thereof for model training;
constructing inputs for training based on the sgRNA sequences in the sample dataset and one or more corresponding epigenetic features;
building a convolutional neural network CNN model;
dividing the sample dataset into a training set and a testing set, training the CNN model using an input matrix and output ground truth of each sample in the training set, and determining the output accuracy of the trained model using the testing set;
terminating the training when the output accuracy meets the requirement, thereby obtaining model parameters that have been trained.

2. The method according to claim 1, wherein the sample dataset comprises at least one of the following: CRISPRoff_tiling dataset, CRISPRoff_genomeA dataset, CRISPRi_intergrate dataset, CRISPRi_genome dataset, CRISPRi_CRISPRoffsource dataset, hCRISPRiV2 dataset, hCRISPRav2 dataset, or CRISPRa_intergrate dataset,
preferably, for a CRISPR/dCas editing system that inhibits gene expression, the sample dataset is CRISPRoff_tiling dataset, and for a CRISPR/dCas editing system that activates gene expression, the sample dataset is hCRISPRiV2 dataset.

3. The method according to claim 2, wherein the CRISPRoff_tiling dataset is based on CRISPRoff screening experiments in HEK293T cells, comprising 520 genes and 111,638 targeting sgRNAs.

4. The method according to claim 1, wherein the input matrix is obtained by:
constructing the DNA sequence of the genomic region associated with the sgRNA sequence in the training sample as a binary matrix according to the base type at each nucleotide position, using one-hot encoding;
constructing the one or more epigenetic features at each nucleotide position as a continuous variable matrix; and
concatenating the binary matrix and the continuous variable matrix as an input matrix for training.

5. The method according to claim 4, wherein the DNA sequence has a length of 40 base-pairs.

6. The method according to claim 4, wherein the DNA sequence comprises an upstream sequence of 9 base-pairs, a protospacer sequence of 20 base-pairs, a PAM sequence of 3 base-pairs, and a downstream sequence of 8 base-pairs-, wherein the protospacer sequence of 20 base-pairs corresponds to the sgRNA sequence in the training sample.

7. (canceled)

8. The method according to claim 1, wherein the CNN model includes five parallel convolution layers and three cascaded fully connected layers behind the convolution layers, the five convolution layers extract features from the input matrix in parallel, and the outputs from each convolution layer are concatenated as inputs to the fully connected layers.

9. (canceled)

10. The method according to claim 8, wherein the CNN model further includes at least one of the following:
a pooling layer between the convolution layers and the fully connected layers, an input layer using a linear activation function, a drop-out function behind the convolution layers, or a drop-out function behind the fully connected layers, optionally the drop-out function has a drop-out rate of 0.4.

11. The method according to claim 1, wherein the CNN model includes a classification model or regression model.

12. The method according to claim 11, further comprising:
in response to the CNN model as a classification model, labelling the output ground truth of each training sample, of which, those with an editing effect greater than the threshold are labelled as “1”, and the remaining ones are labelled as “0”; or
in response to the CNN model as a regression model, labelling an output ground truth of each training sample, with the output ground truth indicating the editing efficiency level of sgRNA.

13. (canceled)

14. The method according to claim 12, wherein the output ground truth includes a phenotype score γ, which is calculated as follows:
phenotype score(γ)=Log₂sgRNA enrichment/fold difference.

15. The method according to claim 1, wherein the one or more epigenetic features include: the distance between transcription start site (TSS) and sgRNA target site, DNA methylation level, RNA expression level and chromosome accessibility.

16. The method according to claim 15, wherein the epigenetic features of DNA methylation level, RNA expression level and chromosome accessibility are quantified by whole-genome bisulfite sequencing data, RNA-seq data, and ATAC-seq data, respectively.

17. (canceled)

18. The method according to claim 1, further comprising executing repeatedly the following process multiple times: dividing the sample dataset into a training set and a testing set, training the CNN model using a input matrix and output ground truth of each sample in the training set, and determining the output accuracy of the trained model using the testing set, wherein the training set and testing set are randomly re-divided for each execution to ensure the stability of the model, optionally the sample dataset is divided into a training set and a testing set at a ratio of 9:1.

19. The method according to claim 8, wherein each of the five convolution layers uses 30 filters, and the kernel size of the convolution layers are 1, 2, 3, 4, and 5, respectively.

20. The method according to claim 8, wherein the three fully connected layers contain 80, 60 and 40 units, respectively.

21. (canceled)

22. A method for predicting the gene editing activity of a CRISPR/dCas sgRNA based on deep learning, comprising:
establishing a prediction model for predicting the gene editing activity of CRISPR/dCas sgRNA based on a sample dataset comprising sgRNAs and editing activity data thereof, one or more epigenetic features, and a convolutional neural network model;
transforming the sequence of the sgRNA to be tested and related epigenetic features thereof into an input matrix suitable for the prediction model, and inputting it into the prediction model to obtain a predicted value of the sgRNA's gene editing activity.

23. The method according to claim 22, wherein the one or more epigenetic features include: the distance between transcription start site (TSS) and sgRNA target site, DNA methylation level, RNA expression level and chromosome accessibility.

24. A computer system for assisting users in predicting the editing activity in gene editing systems, comprising:
one or more processors; and
one or more memories configured to store a series of computer-executable instructions,
wherein, when the series of computer-executable instructions are executed by the one or more processors, the one or more processors are allowed to perform the method according to claim 1.

25. A non-transitory computer-readable storage medium, wherein, a series of computer-executable instructions are stored on the non-transitory computer-readable storage medium, and when the series of computer-executable instructions are executed by one or more computing devices, the one or more computing devices are allowed to perform the method according to claim 1.
